(19) United States
(12) Patent Application Publication
(10) Pub. No.: US 2012/0296380 A1
(43) Pub. Date: Nov. 22, 2012

(54) HINGED BONE SCREW WITH A TULIP BULB CONNECTOR IN A SPINAL FIXATION ASSEMBLY

(76) Inventor: Peter Melott Simonson, Longboat Key, FL (US)

(21) Appl. No.: 13/451,412

(22) Filed: Apr. 19, 2012

Related U.S. Application Data
(60) Provisional application No. 61/479,273, filed on Apr. 26, 2011.

(51) Int. Cl. A61B 17/70; A61B 17/88

(52) U.S. Cl. 606/278; 606/279

(57) ABSTRACT

A spinal fixation assembly using a hinged bone screw with a tulip blob connector, a variable height, hinged bone screw with a tulip bulb connector or both. The bone screws have a post section connected to a screw section by a hinge. In the basic hinged bone screw, a bulb shaped head is formed at the end of the post section opposite the hinge and a tulip bulb connector is attached to the bulb shaped head. For the variable height hinged bone screw, a collet is used with an interior bore that slides over the post section of the bone screw. The collet has a cylindrically shaped sleeve at one end and a bulb shaped head at the other end. The tulip bulb connector is attached to the bulb shaped head of the collet. In both cases, the tulip bulb connector has a cavity for receiving a connector rod and a set screw for firmly attaching said connector rod within said tulip bulb connector cavity.

### CROSS-REFERENCE TO RELATED APPLICATIONS

This application claims priority to U.S. Provisional Application No. 61/479,273, filed on Apr. 26, 2011, the disclosure of which is incorporated herein by reference in its entirety for all purposes.

### BACKGROUND OF THE INVENTION

The present invention relates to orthopedics and spinal surgery and, more particularly, to a hinged bone screw with a tulip bulb connector in a spinal fixation assembly.

Back pain is a commonly reported medical aliment. It is most frequently associated with degenerative changes or fractures in the spinal vertebra. Most of the 30 million U.S. patients who report back pain each year resolve their pain with conservative treatment (e.g., rest and exercise). Nonetheless, approximately 15 percent, or 4.5 million, fail conservative therapy and are left with debilitating pain. Out of these, approximately 500,000 people opt for spinal surgery. In addition to alleviating pain, spinal surgery seeks to minimize damage to adjacent supportive muscle and skeletal components.

Several techniques and systems have been developed for correcting and stabilizing the spine and facilitating a common spinal surgical procedure—spinal fusion. The most widely used systems use a bendable rod that is placed longitudinally along the length of the spine. Such a rod is bent to follow the normal curvature of the spine, whether it is the normal kyphotic curvature for the thoracic region or the lordotic curvature for the lumbar region. In such a procedure, a rod is attached to various vertebrae along the length of the spinal column by a number of bone anchor assemblies. A bone anchor may be a hook that engages the vertebra laminae or a bone screw threaded into the vertebral body. When stabilized, the vertebra is decortified where the outer cortical bone is removed to provide a foundation for bone grafts. Over time, these bone grafts fuse the damaged vertebrae together.

A good example of a traditional rod spinal fixation assembly is the Cotrel-Dubosset/CD Spinal System® sold my Medtronic Sofamor Danek, Inc. As shown in FIG. 1, the CD Spinal System® includes a bone screw with a tulip bulb connector 2 having a top cavity 4 where the spinal rod 6 is placed. The cavity includes a threaded bore into which a set screw 8 is engaged to clamp the rod 6 down. Additional details of this technology can be found in U.S. Pat. No. 5,005,562 to Cotrel. One benefit of the CD Spinal System is that the fixation element is positioned directly beneath the rod. Although the bone screw 10 rotates 12 about 30° from vertical in the lateral direction 14 in the coronal plane 16, it lacks the ability to make spinal adjustments in the dorsal 18, ventral 20 and medial-sagittal 22 planes. Since the CD Spinal System® can only rotate in lateral directions 14 from vertical within the coronal plane 16, any dorsal, ventral and medial vertebral body correction or translation is limited.

In degenerative and deformity cases, the spine is misaligned in either the coronal (scoliosis) and sagittal (kyphosis) planes or both (spondylolisthesis). For such degenerative and deformity cases, the concept of attaching a pre-contoured rod to a deformed spine was used by Luque and Asher (w/wires and cables) for scoliosis, and Edwards (w/threaded connectors) and Steffe (w/threaded screw posts) for spondylolisthesis. For many years, there was no single bone screw spinal fixation assembly that solved all of these problems. When vertebral correction or translation occurred in both the coronal and sagittal planes, it generated such high force loads that bone screw pullout was common. In cases when the bone is strong and healthy, the initial fixation of traditional spinal and orthopedic screws is usually excellent and pullout strength is around 150 N/mm. With degenerative cases, pullout strength falls to about 50-60 N/mm.

To address these shortcomings, a hinged bone screw shown in FIG. 2 was disclosed in U.S. Pat. Nos. 6,309,391 and 7,322,979 to Crandall. Whereas a short screw post enhances bone screw assembly in passive fixation, the long post screw 24 shown in FIG. 2 facilitates simultaneous correction in both the coronal 16 and sagittal 22 planes in active fixation cases. When the long post screw 24 is screwed into the pedicle 26, it offers vertebral body movement 28 in both the coronal 16 and sagittal 22 planes through its hinge 30. In combination with the vertical 32 and rotational ability of Simonson's TSRH® 3D connectors 34, pulling the vertebrae 36 and the spine to the pre-contoured rod 38 via this pivoting post system facilitates simultaneous correction in both the coronal 16 and sagittal 22 planes. By adjusting the vertical height 32 of the TSRH® 3D connectors 34 shown in FIG. 3, the vertebrae 36 can now move in the lateral-sagittal 40, medial-sagittal 42, dorsal-coronal 44 and ventral-coronal 46 directions (FIG. 3). Together, the hinged bone screw of Crandall and TSRH® 3D connectors 34 by Simonson provide a planar rotation of more than 180° from vertical and allows for vertebral body correction or translation 48 in all planes. With such a bone screw fixation assembly, the force load on a bone screw can be reduced by as much as 60%. A commercial example of such a vertebral translation system is the TSRH-3Dx® Multi-Planar Adjusting (MPA™) Screw 50 sold by Medtronic Sofamor Danek Inc. Shown in FIG. 3, the MPA™ Screw 50 repositions the vertebrae 36 in medial-sagittal plane 22 while also placing the vertebrae 36 into a more dorsal-coronal plane 18 toward the rod 38. If necessary, it can also move the spine downward toward the ventral-coronal plane 20. With such vertebral translation 48, the force load on a MPA™ Screw 50 averages between 20-40 N/mm—well below the bone screw pullout strength in degenerative bone.

This principle of direct vertebral translation in multiple planes with respect to the rod is now a powerful tool for deformity and degenerative correction, especially for scoliosis, kyphosis and spondylolisthesis.

### BRIEF SUMMARY OF THE INVENTION

The present invention provides a spinal fixation assembly that facilitates the simultaneous active and passive correction or translation of fractured, degenerative or deformed vertebrae not only in the coronal plane but also in the dorsal-coronal, ventral-coronal, lateral-sagittal and medial-sagittal planes as well.

In one embodiment, the present invention is a hinged bone screw with tulip bulb connector in a spinal fixation assembly. The first component is a bone screw with a hinge. A shaft or post extends from the hinge. The post can be either short or long in length. With its hinge, the bone screw provides vertebral body movement in both the coronal and sagittal planes.

For the basic hinged bone screw with a tulip bulb connector, there is a bulb-shaped head at the end of the post. In turn, this bulb-shaped head fits into the tulip bulb connector. This tulip bulb connector possesses a top cavity where a spinal rod is placed. The cavity includes a threaded bore into which a set screw is engaged to clamp the rod down. This tulip bulb connector, however, only provides rotational vertebral body movement of about 30 degrees in the coronal plane. By combining the hinged bone screw with the tulip bulb connector, the vertebral body can not only move in the coronal and sagittal plane but also laterally and medially. As a result, the present invention in its basic form increases the rotational ability of the bone screw to about 130° from its original 30° from vertical.

A more advanced form of hinged bone screw of the present invention includes a collet—a cylindrically shaped sleeve with a bulb shaped head. The bulb shaped head of the collet is placed into the tulip bulb connector. Both the collet bulb and cylindrical shaft have an interior bore that slides over the bone screw post like a sleeve or collet. By changing the amount of the post covered by this collet, the height of the bone screw vis-à-vis the tulip shaped connector can be varied. In other words, the collet allows for variable height positions along the post. By combining the collet with the hinged bone screw and tulip bulb connector, simultaneous correction in both the coronal and sagittal planes is now possible. If one adjusts the vertical height of the tulip bulb connector with this configuration, the hinged bone screw fixation assembly can now move the vertebrae in the lateral-sagittal, medial-sagittal, dorsal-coronal and ventral-coronal directions, thereby vastly increasing the vertebral body translation abilities.

With such a system, the force load on the hinged bone screw falls between 20-40 N/mm—well below the bone screw pullout strength in degenerative bone. With this hinged bone screw fixation assembly, direct vertebral translation in multiple planes is now possible in deformity and degenerative cases, especially for scoliosis, kyphosis and spondylolisthesis.

### BRIEF DESCRIPTION OF THE DRAWINGS

FIG. 1 shows the planar movements of a prior art bone screw with a tulip bulb connector.

FIG. 2 shows a view of the prior art TSRH-3D® Multi-Planar Adjusting (MPA™) Bone Screw.

FIG. 3 shows the planar movements of the prior art TSRH-3Dx® Multi-Planar Adjusting (MPA™) Bone Screw.

FIG. 4 shows a side view of a hinged bone screw of the present invention attached to a tulip bulb connector.

FIG. 5 shows a side view of a hinged bone screw of the present invention without the tulip bulb connector.

FIG. 6 shows a front view of a hinged bone screw of the present invention without the tulip bulb connector.

FIG. 7 shows the lateral and medial movement of a hinged bone screw of the present invention with a tulip bulb connector.

FIG. 8 shows the coronal rotation movement of the tulip bulb connector along with the lateral and medial movement of a hinged bone screw.

FIG. 9 shows the planar movements of a hinged bone screw with a tulip bulb connector of the present invention.

FIG. 10 shows the variable height, hinged bone screw with a tulip bulb connector of the present invention.

FIG. 11 shows the planar movements of the variable height, hinged bone screw of FIG. 10.

FIG. 12 shows a spondylolisthesis reduction using a variable height, hinged bone screw with a tulip bulb connector along with the CD Horizon® Sextant® II System.

FIG. 13 shows a variable height, hinged bone screw with a tulip bulb connector actively moving the vertebral body in the coronal, sagittal and dorsal planes in conjunction with CD Horizon® Sextant® II System

FIG. 14 shows variable height, hinged bone screws in a spinal fixation assembly moving the vertebral body passively in conjunction with CD Horizon® Longitude® System.

### DETAILED DESCRIPTION OF THE INVENTION

### I. Definitions

“Active Fixation” means moving, fixing and stabilizing the spine, generally used in degenerative and deformity cases.

“Coronal” means a vertical plane that divides the body into a ventral and dorsal section (belly and back) sections. It is also known as the frontal plane.

“Dorsal” refers to a plane that is parallel to or in a direction toward the back.

“Kyphosis” means a curvature of the upper back, also known as hunchback.

“Lateral” means to the side, either left or right.

“Median or Medial” defines a point in the center of the organism that bisects the body vertically through the navel, dividing the body in a left and right side.

“Mid-sagittal” is the mid-line that passes through the navel or spine and all other sagittal planes are parallel to it.

“Passive Fixation” means fixing and stabilizing the spine in, generally, fractured or degenerative cases.

“Pullout Strength” means the force or stress necessary to dislodge an embedded bone screw from a bone.

“Sagittal” refers to a plane that divides the body into right or left parts.

“Scoliosis” means the spine is curved from side to side creating a “S” or “C” shape.

“Spinal Fixation Assembly” means a complete spinal system including bone screws, connectors and rods.

“Spondylolisthesis” means the vertebrae are misaligned by slipping over one another either forwards (anterolisthesis) or backwards (retrolisthesis).

“Ventral” means a plane or direction toward the abdomen or belly.

### I. Hinged Bone Screw with a Tulip Bulb Connector

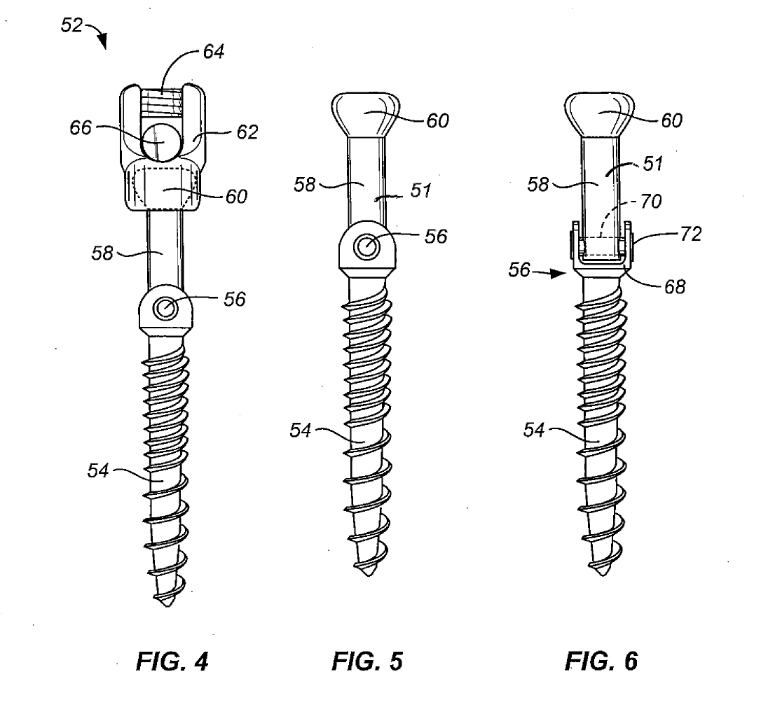

A hinged bone screw with a tulip bulb connector 52 is shown in FIG. 4. It consists of a bone screw 54, a hinge 56, a post 58, a bulb shaped head 60, a tulip bulb connector 62, a set-screw 64 and a rod 66.

FIG. 5 shows the same hinged bone screw 51 as in FIG. 4, but without the tulip bulb connector 62 assembly. In this view, the bulb shaped head 60 of the hinged bone screw 51 can be more clearly seen. FIG. 6 shows a side view of the hinged bone screw 51 where the details of the hinge 56 can be more clearly seen. For this hinge 56, a pin 70 goes through a hole in the post 58 and attaches to both sides of the screw flanges 68. The pin 70 is preferably flared or stamped 72 at both ends to secure it to the hinge. It may also be secured with a screw. The length of the post 58 can be varied. Now referring back to FIG. 4, a tulip bulb connector 62 with a threaded bore is shown into which a set screw 64 is engaged to clamp a connector rod 66 down. While a tulip bulb connector 62 is illustrated, one of skill in the art will recognize that other types of connectors can be used to connect the hinged bone screw 51 to the connector rod 66.

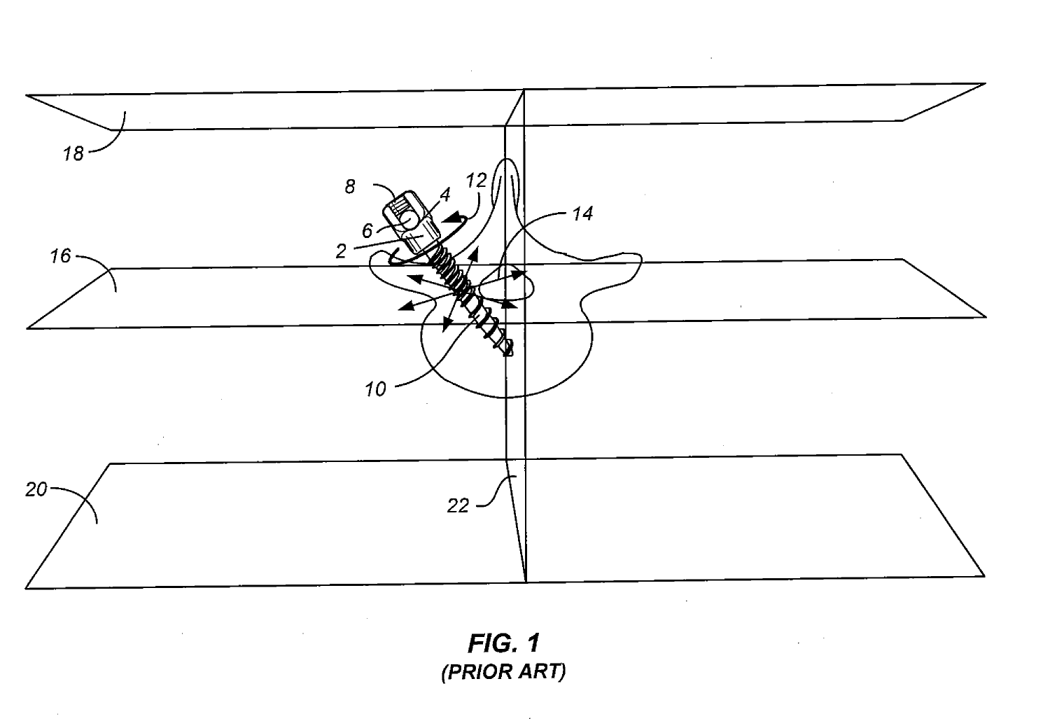

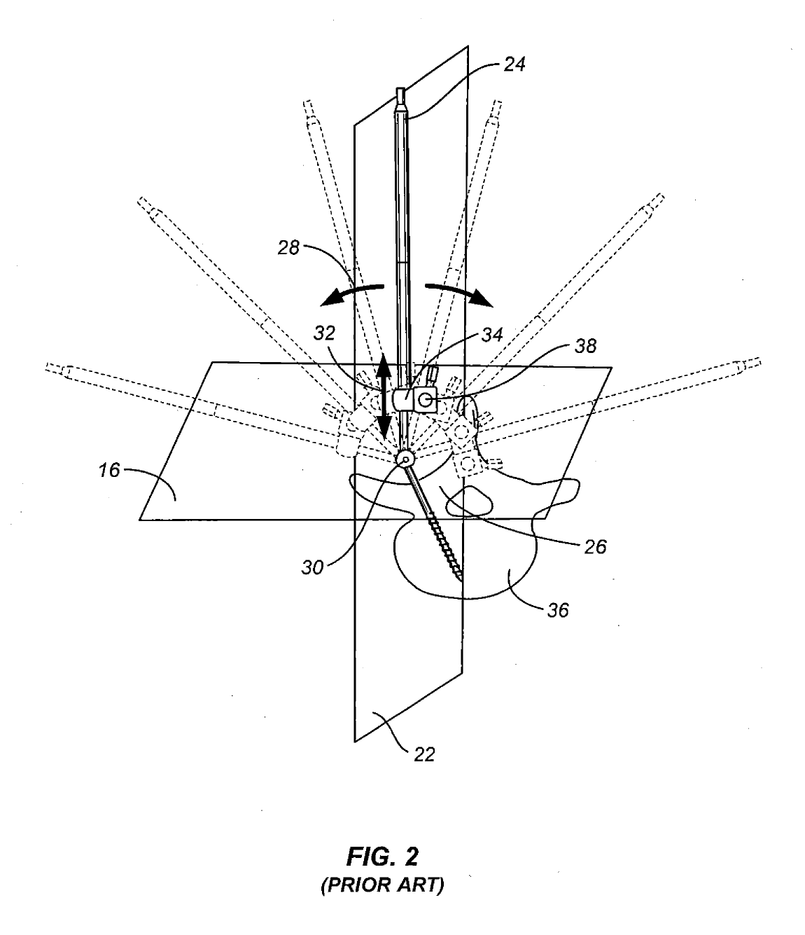

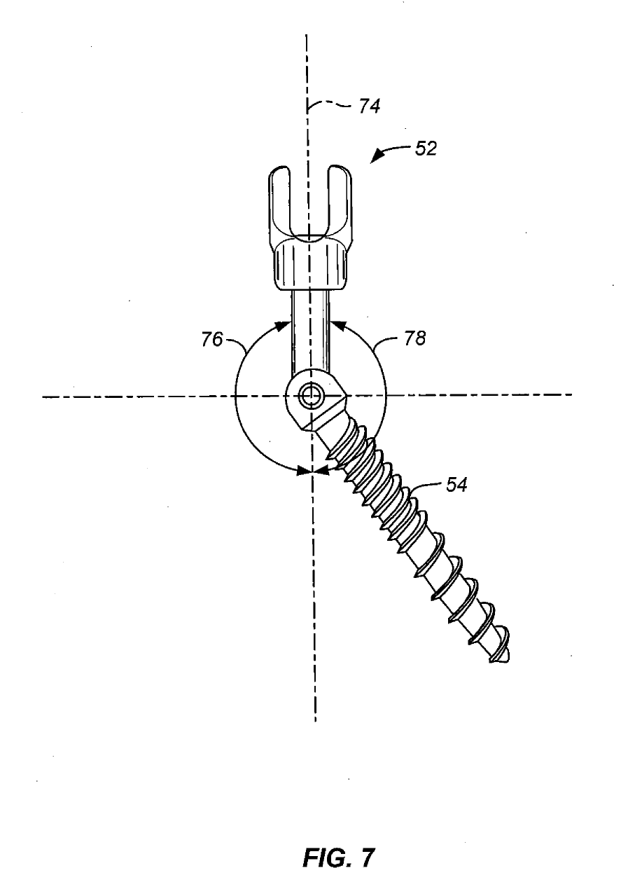

The hinged bone screw with a tulip bulb connector 52 provides additional planar movements as compared with prior art bone screws. Whereas the bone screw with tulip bulb connector 2 shown in FIG. 1 only provides about a 30° lateral 14 movement on either side of vertical in the coronal plane, the hinged bone screw with a tulip bulb connector 52 shown in FIG. 7 now allows the bone screw 54 to move almost 180° from its vertical line 74 in either the lateral 76 and medial 78 directions or both. In either case, it is more rotation than necessary for any normal vertebral body translation.

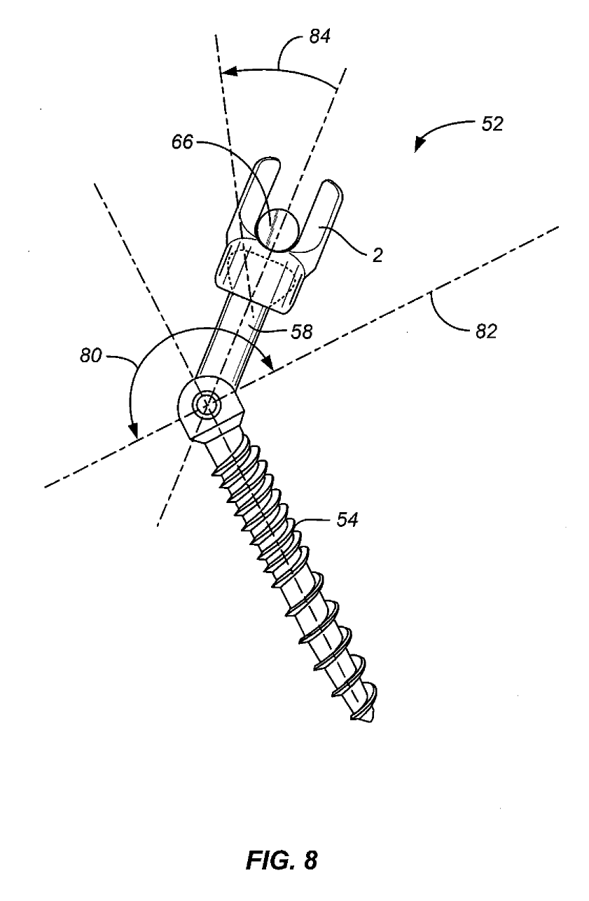

In FIG. 8, the post 58 can also rotate 80 another 180° from its horizontal 82 line allowing both the bone screw 54 and post 58 to rotate simultaneously with respect to one another. As shown earlier in FIG. 1, the prior art bone screw with tulip bulb connector 2 can also rotate 12 in the coronal plane 16. This ability to either tilt 84 the tulip bulb connector 2 of the entire hinged bone screw with tulip bulb connector 52 shown in FIG. 8 further enhances the present invention. When tilted 84, the hinged bone screw with a tulip bulb connector 52 can be positioned directly beneath or closer to the rod 66.

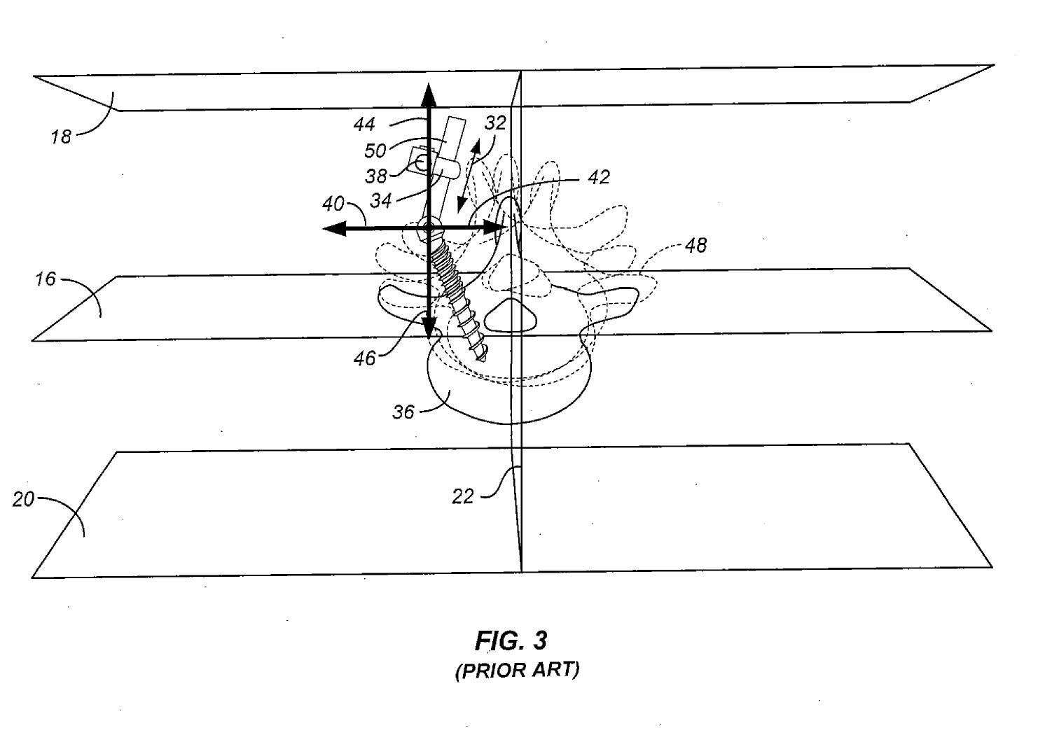

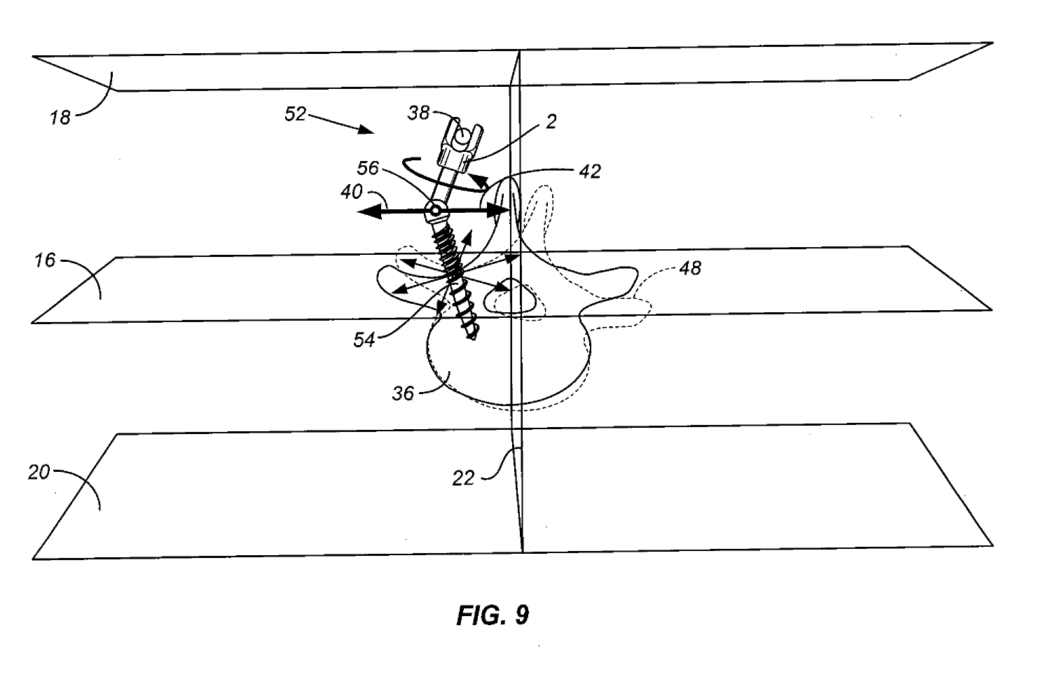

By combining the tulip bulb connector 2 and hinge 56 shown in FIG. 9, the hinged bone screw with tulip bulb connector 52 of the present invention can now move a vertebrae 36 in both the coronal 16 and sagittal 22 planes in either the lateral 40 and medial 42 directions or both. Similar to the prior art TSRH-3Dx® Multi-Planar Adjusting (MPA™) Screw 50 shown in FIG. 3, the hinged bone screw with tulip bulb connector 52 can now add vertebral translation 48 to the spine in the sagittal plane 22 and, most important, medially 42 toward the spinal rod 38. With the present invention, it is estimated that the vertebral translation of a hinged bone screw with a tulip bulb connector 52 has increased from 30° to 130° giving the original tulip bulb connector 2 a new flexibility it never possessed. With the present hinged bone screw with tulip bulb connector 52 invention, the force load on the bone screw 54 also falls during translation.

The present invention, therefore, also helps reduce bone screw pullout.

### II. Variable Height, Hinged Bone Screw with Tulip Bulb Connector

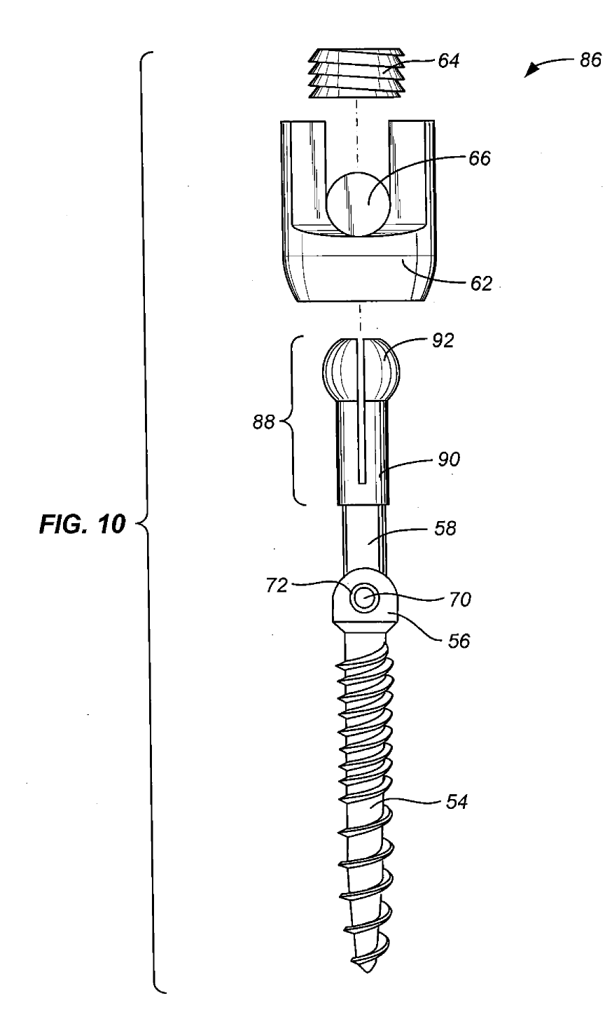

The hinged bone screw with tulip bulb connector 52 shown in FIG. 9 can be limited in its ability to move the vertebrae 36 in either the dorsal or ventral directions, thereby, restricting the movement in the dorsal 18 or ventral 20 planes. To overcome this limitation, a further preferred embodiment of the present invention is shown in FIG. 10. The variable height, hinged bone screw with tulip bulb connector 86 shown in FIG. 10 includes a bone screw 54, a hinge 56, a post 58 and a collet 88. The collet 88 consists of a cylindrically shaped sleeve 90 at its lower end and a bulb shaped head 92 at its upper end. The bulb shaped head 92 is, in turn, placed into a tulip bulb connector 62. Both the bulb shaped head 92 and cylindrically shaped sleeve 90 have an interior bore that slides over the post 58. A more full description of the configuration and interaction of the collet 88 with the tulip shaped connector 62 is provided in Applicant's pending U.S. patent application Ser. No. 12/731,116, which is hereby incorporated by reference. The hinge 56 of the variable height, hinged bone screw with tulip bulb connector 86 has the same construction as the hinge 56 of the previous hinged bone screw with tulip bulb connector 52 (see, FIG. 4-6). More specifically, a pin 70 goes through a hole in the post 58 and attaches to both sides of flanges at the upper end of the bone screw 54. The pin 70 is preferably flared or stamped 72 at both ends to secure it to the hinge 56. It may also be secured with a screw. Although the length of the post 58 may be varied, the collet 88 now provides the ability to vary the height of the tulip shaped connector 62 vis-a-vis the hinge 56 without having to switch out or change the post 58. Like the tulip bulb connector 62 in the earlier hinged bone screw with tulip bulb connector 52 embodiment (see, FIGS. 4-6), the tulip bulb connector 62 in the variable height, bone screw with tulip bulb connector 86 embodiment also has a threaded bore into which a set screw 64 is engaged to clamp a connector rod 66 down.

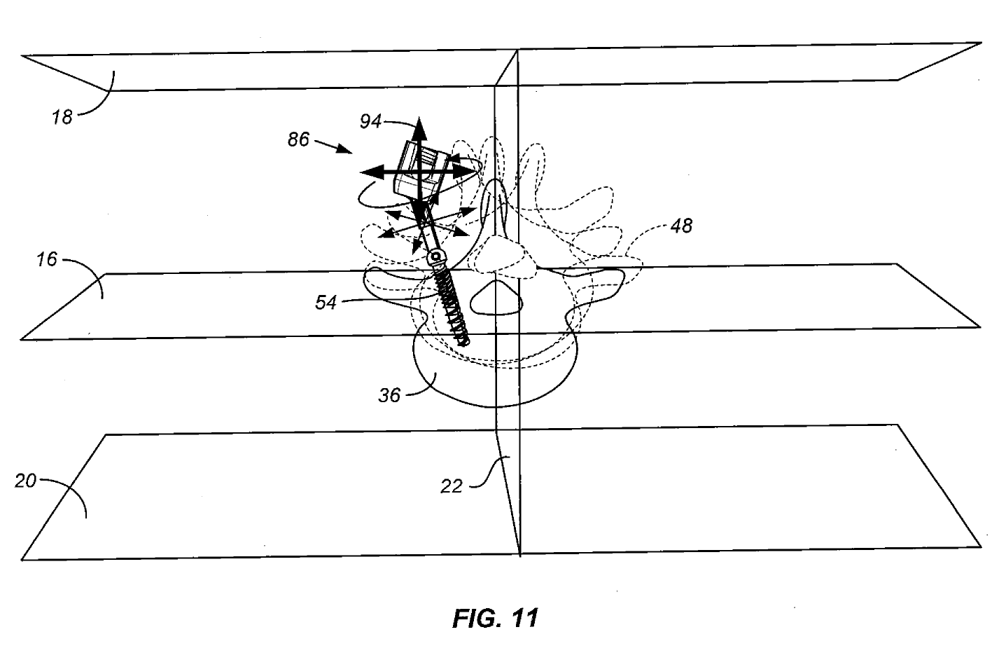

By changing the amount of the bone screw post 58 covered by the collet 88 in FIG. 10, one can vary the vertical height 94 (FIG. 11) of the tulip shaped connector 62 vis-a-vis the hinge 56 to adjust its position in either the dorsal 18 or ventral 20 planes. The variable height, hinged bone screw with tulip bulb connector 86 of the present invention allows the bone screws 54 to be set at different axes vis-à-vis the connecting rods 66. It can also be set at different vertebral body heights 48 vis-à-vis such rods to engage a fixed cylindrical rod in any degree of angular orientation or direction. Similar to the prior art TSRH-3Dx® Multi-Planar Adjusting (MPA™) Screw 50 referred to in FIG. 3, the variable height, hinged bone screw with tulip bulb connector 86 now provides direct vertebral height 48 translation in multiple planes for deformity and degenerative cases and, especially, for scoliosis, kyphosis and spondylolisthesis. It also does so at reasonable and safe bone screw stress loads.

The components of both the hinged bone screw with tulip bulb connector and the variable height, hinged bone screw with tulip bulb connector are preferably made from metals such as stainless steel, titanium, cobalt chromium, nickel-titanium alloys or other suitable high strength materials. Such components may also be made of polymer materials such as PEEK (polyether ether ketone) or carbon fiber-reinforced polymers where a high strength-to-weight ratio allows reduced size.

### III. Active Fixation Example

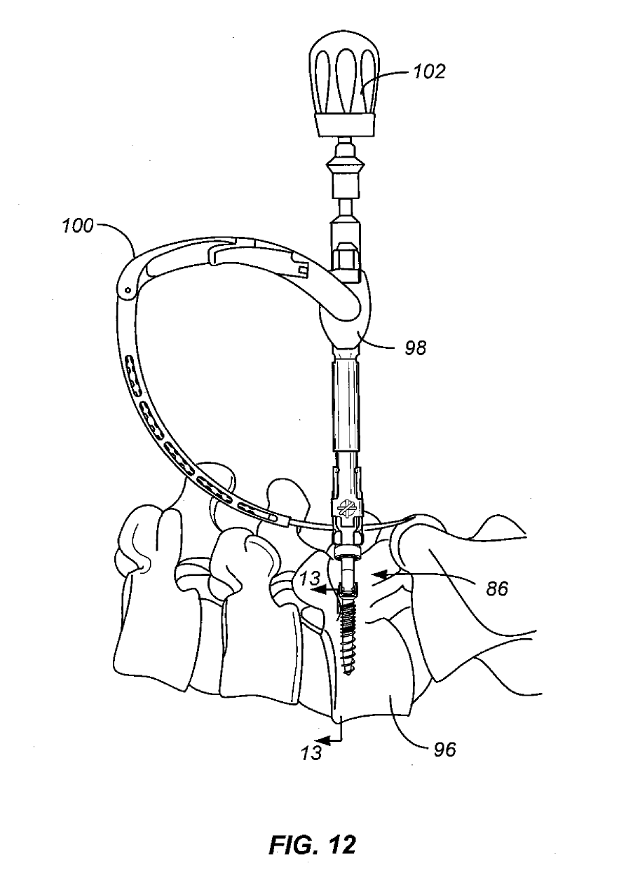

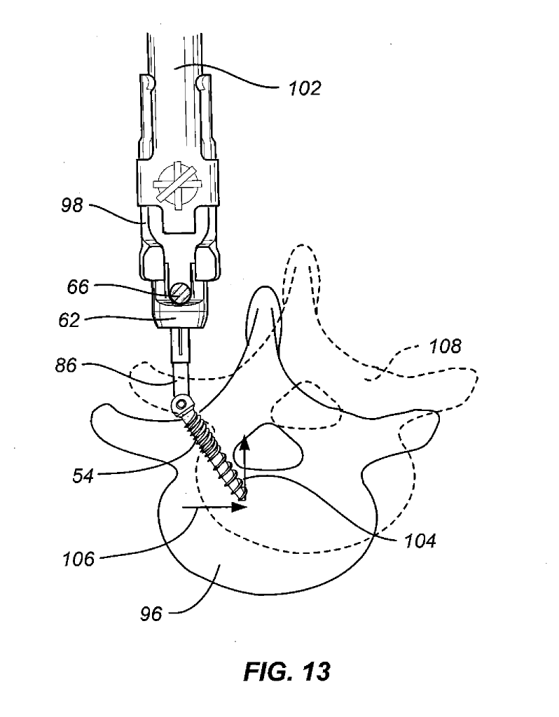

A spinal fixation assembly incorporating the hinged bone screw with tulip bulb connector of the present invention is illustrated in FIG. 12 involving a spondylolisthesis reduction. During active fixation in this example, the fixation assembly can be used to realign a misaligned or deformed spine to a more natural curvature. As shown in FIG. 12, a vertebrae 96 is misaligned in both coronal and sagittal planes. A spinal fixation assembly 86 having a variable height, hinged bone screw with a tulip bulb connector of the present invention is attached to the vertebrae 96. In this case, the CD Horizon® Sextant® II System 98 sold by Medtronic Sofamor Danek, Inc. is connected to the variable height, hinged bone screw with a tulip bulb connector and used to perform the correction of the misaligned vertebrae 96. The CD Horizon® Sextant® II System 98 uses an arc arm 100 for leverage to minimize the stress on the bone-screw interface during reduction. Such an instrument facilitates spondylolisthesis correction by using top-loading screws to reduce the deformity. The CD Horizon® Sextant® II System 98 locks onto the tulip bulb connector of the variable height, hinged screw. It then engages the driver 102 of the CD Horizon® Sextant® II System 98. With the new pivoting and variable height ability of the variable height, hinged bone screw with tulip bulb connector spinal fixation assembly 86 shown in FIG. 13, the coronal and sagittal misalignment can be properly corrected without undue load stress on the bone screw 54. Since, in this example, coronal and sagittal misalignment occurs at a single vertebrae 96, the variable height, hinged bone screw with tulip bulb connector spinal fixation assembly 86 can reduce or move this vertebrae 96 either dorsally 104 in the coronal plane, medially 106 in the sagittal plane or both. This coronal and sagittal plane reduction process can be performed simultaneously to evenly spread the stress of the reduction throughout the spinal fixation assembly 86. With only a few turns of the driver 102, the present invention provides more accurate and precise force load during vertebral translation, thereby, avoiding possible vertebral fracture and bone screw 54 pullout. With the variable height, hinged bone screw with tulip bulb connector spinal fixation assembly 86, both coronal and sagittal plane reduction can proceed slowly and accurately to align the vertebrae 96 into a natural and neutral spinal position. In this example, the CD Horizon® Sextant® II System 98 slides a pre-contoured rod 66 through the tulip bulb connectors 62 to secure the realigned vertebrae 108 in their proper and natural position. If properly performed, such vertebral translations may alleviate nerve compression and pain caused by such deformities as scoliosis, kyphosis, and spondylolisthesis.

### III. Passive Fixation Example

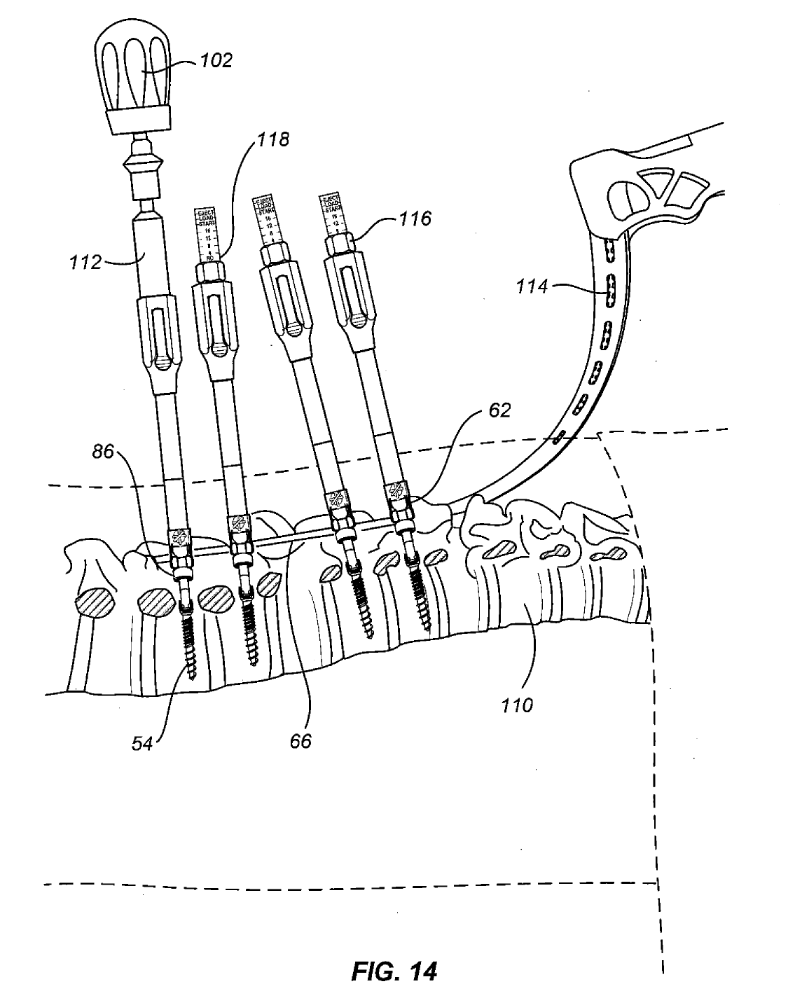

A good example of passive spinal fixation is illustrated in FIG. 14. During passive fixation, a fixation assembly such as the variable height, hinged bone screw with tulip bulb connector spinal fixation assembly 86 is used to stabilize the spine 110. When stabilized, the vertebra is then decortified (i.e., the outer cortical bone is removed) to provide a foundation for bone grafts. Over time, these bone grafts fuse the damaged vertebrae together while the passive fixation assembly continues to support and stabilize the spine.

In this example, the CD Horizon® Longitude® 112 sold by Medtronic Sofamor Danek, Inc. is used to place percutaneous screws and rods at multiple levels of vertebrae. As shown in FIG. 14, a key element of this instrument set are a free-hand or steerable rod inserter 114 and reduction screw extenders 116 that allow for tactile, freehand rod passage through the large holes at the base of screw extenders 116.

As shown in FIG. 14, a set of extenders 116 is placed on several variable height, hinged bone screw with tulip bulb connector spinal fixation assemblies 86. When only using tulip bulb connectors 62, these extenders 116 are moved by a driver 102 in stages because the complete reduction of one extender 116 without reducing the others will cause the rod 66 to put a strong force on the other extenders 116. It is important not to over-rotate the extenders 116 when using the tulip bulb connector 62. When the extender 116 is in the RD position 118, advancing any further will place unneeded pressure on the tulip bulb connector 62. With the variable height, hinged bone screw with tulip bulb connector spinal fixation assemblies 86, this pressure is distributed throughout the assembly. As a result, reduction in any plane can be performed simultaneously with a lower probability of bone screw 54 pullout.

In the foregoing specification, the invention has been described with reference to specific preferred embodiments and methods. It will, however, be evident to those of skill in the art that various modifications and changes may be made without departing from the broader spirit and scope of the invention. For example, while the hinged bone screw with tulip bulb connector spinal fixation assembly and the variable height, hinged bone screw with tulip bulb connector spinal fixation assembly has been described for vertebral translation, those of skill in the art will recognize that alternative uses in orthopedics and spinal surgery are possible. Accordingly, the specification and drawings are to be regarded in an illustrative, rather than restrictive, sense; the invention being limited only by the appended claims.

What is claimed is:

1. A spinal fixation assembly comprising:
a bone screw having a post section connected to a screw section by a hinge;
a bulb shaped head at the end of said post section opposite said hinge;
a connector attached to said bulb shaped head;
wherein said connector has a cavity for receiving a connector rod and a set screw for firmly attaching said connector rod within said connector cavity.

2. The spinal fixation assembly of claim 1 wherein said hinge includes a pin that connects said post section to said screw section by fitting through a hole in said post section and one or more flanges of said screw section.

3. A method for vertebral translation comprising the steps of:
selecting a spinal fixation assembly comprising a plurality of bone screws, each having a post section connected to a screw section by a hinge, a bulb shaped head at the end of said post section opposite said hinge, a connector attached to said bulb shaped head,
wherein said connector has a cavity for receiving a connector rod and a set screw for firmly attaching said connector rod within said connector cavity;
attaching bone screws from said spinal fixation assembly to a plurality of vertebra;
inserting a connector rod through the cavities in said connectors so that the plurality of vertebra are either maintained in proper alignment or urged toward properly alignment;
tightening down a set screw on each connector to firmly attached said connector rod to said connector.

4. A spinal fixation assembly comprising:
a bone screw having a post section connected to a screw section by a hinge;
a collet with an interior bore that slides over the post section of said bone screw;
a connector attached to said collet;
wherein said connector has a cavity for receiving a connector rod and a set screw for firmly attaching said connector rod within said connector cavity.

5. The spinal fixation assembly of claim 4 wherein said collet has a cylindrically shaped sleeve at one end and a bulb shaped head at the other end.

6. A method for vertebral translation comprising the steps of:
selecting a spinal fixation assembly comprising a plurality of bone screws, each having a post section connected to a screw section by a hinge, a collet with an interior bore that slides over the post section of said bone screw, a connector attached to said collet;
wherein said connector has a cavity for receiving a connector rod and a set screw for firmly attaching said connector rod within said connector cavity;
attaching bone screws from said spinal fixation assembly to a plurality of vertebra;
inserting a connector rod through the cavities in said connectors so that the plurality of vertebra are either maintained in proper alignment or urged toward proper alignment;
tightening down a set screw on each connector to firmly attached said connector rod to said connector.
